(19) United States
(12) Patent Application Publication
(10) Pub. No.: US 2025/0157279 A1
(43) Pub. Date: May 15, 2025

(54) AUTOMATED MEDICATION UNITS DISPENSING DEVICE AND ASSOCIATED METHOD

(71) Applicant: Chronomed Inc., Longueuil (CA)

(72) Inventors: Frédéric Droulers, Longueuil (CA); Maude Laforest, Longueuil (CA); Samuel Whittom, Longueuil (CA)

(21) Appl. No.: 18/726,887

(22) PCT Filed: Jan. 6, 2023
(86) PCT No.: PCT/CA2023/050009

§ 371 Date: Jul. 5, 2024

Related U.S. Application Data
(60) Provisional application No. 63/266,538, filed on Jan. 7, 2022.

(51) Int. Cl. G07F 17/00

(52) U.S. Cl. CPC G07F 17/0092

(57) ABSTRACT

A medication units dispensing device having at least one cassette and a robotic subsystem including a motorized system and a robotic head where each cassette stores medication units of a medication drug and includes a housing, a slotted disk positioned at a lower section of the housing with slots being defined in a periphery of the slotted disk and a coupling wheel positioned under the housing and engaged with the slotted disk to rotate simultaneously with the slotted disk and where the robotic head is removably engageable with the cassette and is movable about at least an x-axis, a y-axis and a z-axis by the motorized system. The robotic head includes a motorized activation wheel engaged with the coupling wheel of the cassette when the robotic head is engaged to the cassette and an actuator for rotating the activation wheel. A method for dispensing medication units is also provided.

### CROSS-REFERENCE TO RELATED APPLICATIONS

This application claims priority under 35USC § 119 (e) of U.S. provisional patent application(s) 63/266,538, the specification of which being hereby incorporated by reference.

### TECHNICAL FIELD OF THE INVENTION

The present invention relates to the field of automated dispensers for the pharmaceutical industry. More particularly, it relates to a compact automated device and a corresponding method for the packaging of medication units, each package being associated to a corresponding client.

### BACKGROUND

In the pharmaceutical field, it is common for professionals, such as pharmacists or the like, to use a manual method for the preparation of medication packages in accordance with prescriptions of patients. For example and without being limitative medication packages can be provided via different types of containers such as, for example and without being limitative, vials, packets, multi-dose blisters, etc. One skilled in the art will understand that the preparation of medication packages in accordance with prescriptions of patients can include several types of preparations such as, for example and without being limitative, the preparation of packets or multi-dose blisters for patients required to take a plurality of different medications and/or for the preparation of single vials each containing a specific quantity of medication for a patient. One skilled in the art will also understand that the medications can be in different forms such as, for instance, in tablet form, capsule form or the like.

The above-mentioned manual method requires the pharmacist to manually fill the container(s) or container sections of the associated medication package with the appropriate medications, for each patient. This method is very simple, flexible and requires minimal counter space, as there is no associated machine or specific material required. This method however suffers from several drawbacks, primarily related to human resources management associated with the need of numerous skilled professionals to perform the associated tasks, which is undesirable, especially in periods of shortage of skilled labor. Other drawbacks relate to possible contamination and/or the possible inaccuracies in the preparation of the medication packages stemming from human errors, which can have serious consequences for a patient ingesting improper medication or dosage of medication or create additional work downstream during verification and quality control of the production.

In order to alleviate some of the above-mentioned problems, medication dispensers which can be used to perform automated preparation of the medication packages for the above-mentioned patients required to take a plurality of different medications are also known in the art. Known, medication dispensers however also tend to suffer from drawbacks, which often makes them incompatible with the needs of professionals or businesses which would benefit from medication dispensers. For example and without being limitative, medication dispensers often carry a high acquisition cost, which is unsuited to the financial resources and need of pharmacies having lower prescription volumes. The medication dispensers also often tend to have a large footprint, which is incompatible with the existing space available in the section of the pharmacy for receiving such apparatus and/or to be destined to perform automated preparation of the medication packages for a specific container type, therefore requiring numerous apparatuses to be used to provide preparation of the medication packages for numerous container types, thereby exacerbating the acquisition cost and footprint of the combination of apparatuses.

In view of the above, there is a need for an improved automated medication dispensing device and/or automated method for medication dispensing for performing automated preparation of the medication packages which would be able to overcome or at least minimize some of the above-discussed prior art concerns, by providing constant quality and productivity in a compact space.

### SUMMARY OF THE INVENTION

[In accordance with a first general aspect, there is provided a medication units dispensing device comprising: a cassette storage area for storing at a plurality of cassettes; the plurality of cassettes stored in the cassette storage area, a robotic subsystem control electronics controlling the operation of the robotic subsystem; and a system controller issuing system commands communicated to the control electronics. Each one of the plurality of cassettes stores medication units of a medication drug and includes a housing, a chute defining an outlet channel of the housing, a slotted disk positioned at a lower section of the housing with slots being defined in a periphery of the slotted disk to receive medication units being guided therein by gravity and a coupling wheel positioned under the housing and engaged with the slotted disk to rotate simultaneously with the slotted disk. The slotted disk is rotatable to move the medication units received in the slots towards the outlet channel of the chute and individually dispense the corresponding medication units through the chute. The robotic subsystem includes a motorized system and a robotic head movable about at least an X-axis, a Y-axis and a Z-axis by the motorized system. The robotic head is removably engageable with each one of the plurality of cassettes to remove a corresponding one of the cassettes from the cassette storage area and move the corresponding one of the cassettes to at least one target location where at least one of the medication units can be dispensed in a specific package or a specific section of the package. The robotic head includes a motorized activation wheel engaged with the coupling wheel of the corresponding one of the cassettes when the robotic head is engaged to the corresponding one of the cassettes and an actuator for rotating the activation wheel to dispense at least one of the medication units at the at least one target location.

In an embodiment, the robotic subsystem is configured to move the corresponding one of the cassettes to a plurality of target locations where at least one of the medication units can be dispensed in a specific package or a specific section of the package.

In an embodiment, each one of the plurality of cassettes includes a cassette locking member and the robotic head includes a head locking mechanism activable to be secured with the cassette locking member of corresponding one of the cassettes, the motorized activation wheel being engaged with the coupling wheel of the corresponding one of the cassettes when the cassette locking mechanism is secured with the cassette locking member thereof.

In an embodiment, the cassette locking member includes a magnetic member and the head locking mechanism includes an electromagnet activable to be secured against the magnetic member of the corresponding one of the cassettes for removably coupling the robotic head to the corresponding one of the cassettes.

In an embodiment, the cassette storage area includes at least one cassette support rack section configured to support at least a subset of the plurality of cassettes, with the subset of the plurality of cassettes defining an array of cassettes oriented according to a similar orientation.

In an embodiment, the cassette storage area includes at least two cassette support rack sections each configured to support at least a subset of the plurality of cassettes defining an array of cassettes, thereby defining at least two arrays of cassettes, with the cassettes of each one of the least two arrays of cassettes having a different orientation, and wherein the robotic head includes a rotation mechanism rotating a body of the robotic head so the body can face a specific direction for engaging the corresponding of the cassettes being part of any one of the least two arrays of cassettes.

In an embodiment, each one of the plurality of cassettes includes a hooked shaped protrusion projecting outwardly from a rear surface of the housing and wherein each cassette support rack section of the cassette storage area includes a cassette receiving wall having a plurality of slots defined therein, the plurality of slots each being sized and shaped to receive the hooked shaped protrusion of one of the plurality of cassettes and to support the one of the plurality of cassettes onto the cassette support rack section.

In an embodiment, the robotic subsystem is configured to position the corresponding one of the cassettes at a position along the Z-Axis minimizing rebounds of the at least one of the medication units being dispensed from the corresponding one of the cassettes, for each target location.

In an embodiment, the robotic head includes an optical sensor positioned below the chute of the corresponding one of the cassettes engaged with the robotic head, the optical sensor allowing counting of a delivered quantity of the at least one of the medication units dispensed from the corresponding one of the cassettes, through the chute.

In an embodiment, the chute of each one of the plurality of cassettes is sized, shaped and configured to minimize rebounds of the at least one of the medication units being dispensed therefrom.

In accordance with another general aspect, there is also provided an automated method for medication units dispensing. The method includes the steps of:
a) engaging a cassette stored in a cassette storage area with a robotic head of a robotic subsystem and retrieving the cassette from the cassette storage area;
b) moving the cassette to a first spatial target location for dispensing at least one medication unit to a specific package or section of a specific package;
c) activating the cassette at the first target location by rotating an activation wheel of the robotic head, which in turn rotates a coupling wheel and a slotted disk of the cassette, to dispense a quantity of medication units corresponding to a requested quantity for the specific package or section of the specific package;
d) repeating steps b) and c) with the selected cassette for additional target locations, until all target locations corresponding to at least one specific package or section of at least one specific package requiring a certain quantity of the medication units in the cassette are dispensed;
e) returning the cassette to the cassette storage area using the robotic head and disengaging the cassette from the robotic head; and
f) repeating steps a) to e) for additional cassettes storing medication units to be dispensed in the specific at least one specific package or section of at least one specific package.

In an embodiment, the method includes the further step of receiving and analyzing a command message including instructions relative to one or more cassettes including medication units to be dispensed, a package type associated with at least one spatial target position and one or more quantities of medication units to be dispensed at each spatial target locations corresponding to the specific package or section of the specific package.

In an embodiment, the method further comprises the step of verifying the dispensed quantity of medication units at each target locations using an optical of the robotic head.

In an embodiment, the method further comprises the step of activating a head locking mechanism of the robotic head to secure the head locking mechanism with a cassette locking member of the cassette, the motorized activation wheel being engaged with the coupling wheel of the cassette when the cassette locking mechanism is secured with the cassette locking member.

In an embodiment, the step of activating a head locking mechanism of the robotic head to secure the head locking mechanism with a cassette locking member of the cassette, includes activating an electromagnet of the robotic head to secure the electromagnet against a magnetic member of the cassette.

In an embodiment, the method further comprises rotating a body of the robotic head so the body can face a specific direction for engaging the cassettes of a corresponding one of at least two arrays of cassettes of the cassette storage area, each array including a subset of cassettes oriented in a specific orientation, with the orientation being different for each array.

In an embodiment, the method further comprises positioning the cassette along the Z-axis, in at least one of the first target location and the additional target location, to minimize rebounds of the medication unit being dispensed therefrom.

In an embodiment, the step of retrieving the cassette from the cassette storage area includes moving the cassette with the robotic head to disengage a hooked shaped protrusion projecting outwardly from a rear surface of a housing of the cassette from a slot of a cassette receiving wall of the cassette storage area.<

In an embodiment, the step of returning the cassette to the cassette storage areal includes moving the cassette with the robotic head to engage the hooked shaped protrusion projecting outwardly from the rear surface of the housing of the cassette into the slot of a cassette receiving wall of the cassette storage area.

### BRIEF DESCRIPTION OF THE DRAWINGS

Other objects, advantages and features will become more apparent upon reading the following non-restrictive description of embodiments thereof, given for the purpose of exemplification only, with reference to the accompanying drawings in which:

FIG. 1 is block diagram showing the components and subsystems of the medication dispensing device, in accordance with an embodiment.

FIG. 2 is a perspective view of the medication dispensing device of FIG. 1, shown partially without the envelope and cassette support rack section, in order to favor the visibility of the components of the device.

FIG. 3 is a front perspective view of a cassette receiving wall of the medication dispensing device of FIG. 1 including a plurality of cassettes mounted thereon.

FIG. 4 is an enlarged perspective view of a rear section of the cassette receiving wall of the storage section of FIG. 3, shown with a single cassette mounted thereon.

FIG. 5 is an exploded view of the cassette of FIG. 4.

FIG. 6 is a cross-section view of the cassette of FIG. 4.

FIG. 7 is a close-up perspective view of the robotic subsystem of the medication dispensing device shown in FIG. 1.

FIG. 8 is a close-up perspective view of the robotic head of the robotic subsystem of FIG. 7.

FIG. 9 is a block diagram of the components and communication of data between the components for acquisition of prescription data for controlling the operation of the medication dispensing device, in accordance with an embodiment.

FIG. 10 is a block diagram of the components and communication of data between the components for acquisition of prescription data for controlling the operation of the medication dispensing device, in accordance with an alternative embodiment allowing character recognition from a prescription document to acquire patient prescription data.

FIG. 11 is a flowchart presenting the steps of a method for performing medication dispensing, in accordance with an embodiment.

### DETAILED DESCRIPTION

In the following description, the same numerical references refer to similar elements. The embodiments, geometrical configurations, materials mentioned and/or dimensions shown in the appended figures or described in the present description are embodiments only, given solely for exemplification purposes.

Moreover, although the embodiments of the medication dispensing device and corresponding parts thereof consist of certain geometrical configurations as explained and illustrated herein, not all of these components and geometries are essential and thus should not be taken in their restrictive sense. It is to be understood, as also apparent to a person skilled in the art, that other suitable components and cooperation thereinbetween, as well as other suitable geometrical configurations, may be used for the medication dispensing device, as will be briefly explained herein and as can be easily inferred herefrom by a person skilled in the art. Moreover, it will be appreciated that positional descriptions such as “above”, “below”, “left”, “right” and the like should, unless otherwise indicated, be taken in the context of the figures and should not be considered limiting.

Moreover, although the associated method includes steps as explained and illustrated herein, not all of these steps are essential and thus should not be taken in their restrictive sense. It will be appreciated that the steps of the method for performing medication dispensing described herein may be performed in the described order, or in any suitable order.

To provide a more concise description, some of the quantitative and qualitative expressions given herein may be qualified with the terms “about” and “substantially”. It is understood that whether the terms “about” and “substantially” are used explicitly or not, every quantity or qualification given herein is meant to refer to an actual given value or qualification, and it is also meant to refer to the approximation to such given value or qualification that would reasonably be inferred based on the ordinary skill in the art, including approximations due to the experimental and/or measurement conditions for such given value.

The term “computing device” is used to encompass computers, servers and/or specialized electronic devices which receive, process and/or transmit data. “Computing devices” are generally part of “systems” and include processing means, such as microcontrollers and/or microprocessors, CPUs or are implemented on FPGAs, as examples only. The processing means are used in combination with storage medium, also referred to as “memory” or “storage means”. Storage medium can store instructions, algorithms, rules and/or data to be processed. Storage medium encompasses volatile or non-volatile/persistent memory, such as registers, cache, RAM, flash memory, ROM, as examples only. The type of memory is of course chosen according to the desired use, whether it should retain instructions, or temporarily store, retain or update data.

One skilled in the art will therefore understand that each one of such computing device typically includes a processor (or multiple processors) that executes program instructions stored in the memory or other non-transitory computer-readable storage medium or device (e.g., solid state storage devices, disk drives, etc.). The various functions, modules, services, units or the like disclosed hereinbelow can be embodied in such program instructions, and/or can be implemented in application-specific circuitry (e.g., ASICs or FPGAs) of the computing devices. Where a computer system includes multiple computing devices, these devices can, but need not, be co-located. In some embodiments, a computer system can be a cloud-based computing system whose processing resources are shared by multiple distinct business entities or other users.

It should be appreciated by those skilled in the art that any of the block diagrams shown in the appended figures represents conceptual views of illustrative circuitry embodying the principles disclosed herein. Similarly, it will be appreciated that any flow charts and transmission diagrams, and the like, represent various processes which may be substantially represented in computer readable medium and so executed by a computer or processor, whether or not such computer or processor is explicitly shown.

Broadly described, the present application is directed to a medication dispensing device having specific components and being specifically designed to allow a high productivity and versatility, while having a minimal footprint and overall weight (which can allow on-counter installation), and which provides no cross-contamination during dispensing of medication units.

More particularly, the medication dispensing device comprises a plurality of removable cassettes stored in a storage area, robotics for individually loading each one of the cassettes into the cassette storage areas. As will be described in more details below, the robotics individually handling each one of the removable cassettes includes all the necessary components mounted thereon for manipulating a selected cassette to dispense one or more medication units from the cassette directly to one or more packages (without requiring engagement of the cassette with additional components of the device). Il will be understood that the one or more packages can include multiple packages of the same type or different package types. The packages can be placed or exposed through one or more interfaces such as, but not limited to, doors, tables, conveyors or drawers. The interfaces where medication units may be directly dispensed out of the envelope can be accessible through, but not limited to, chutes or holes.

As will be described in more details below, the one or more cassettes may be stored in the cassette storage area via a human interface or via robotics (which can be the same robotics used for dispensing of the medication units or different robotics).

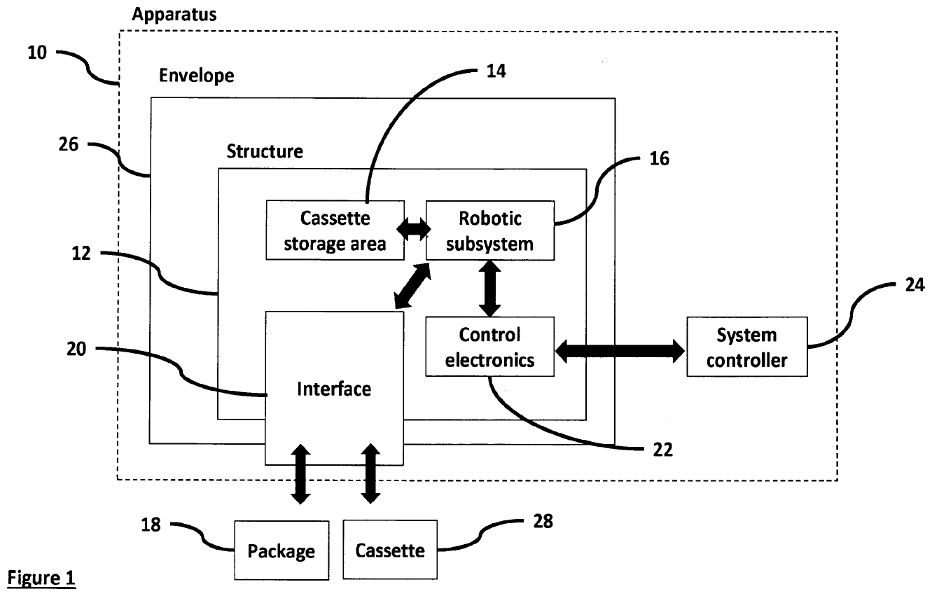

Referring to FIGS. 1 to 3, there is shown a medication dispensing device 10 in accordance with an embodiment. In the embodiment shown, the device 10 comprises a structure 12, a cassette storage area 14, a robotic subsystem 16, one or more packages 18, an interface 20, control electronics 22, a system controller 24 and an external envelope 26.

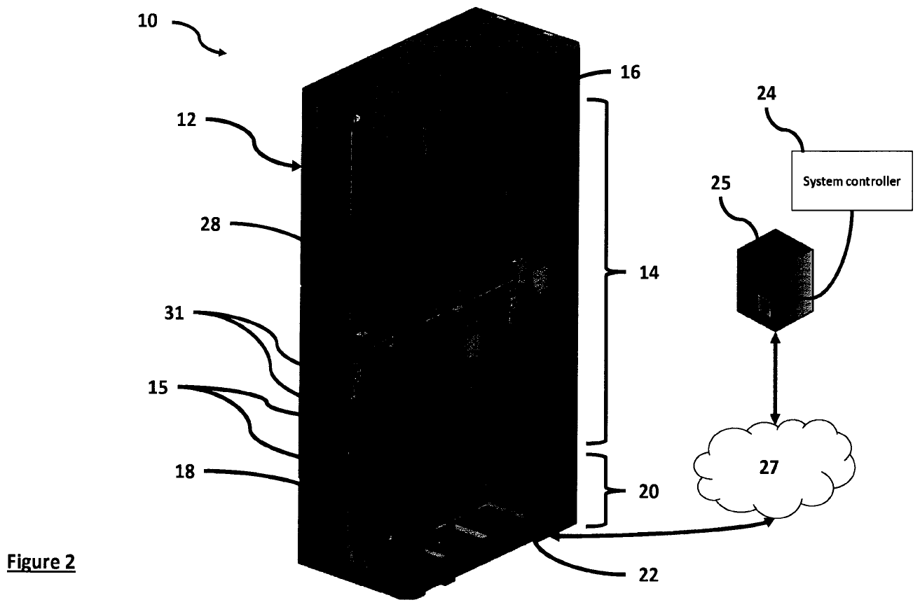

The external envelope 26 surrounds the structure 12 and all other physical subsystems, thereby providing a rigid cover sealing the device 10 from external disturbance. FIG. 2 shows the device with a section of the external envelope 26 removed (i.e. two panes of the external envelope 26 being removed). One skilled in the art would understand that sections of the external envelope 26 being removed are only provided to facilitate understanding of the device and that, in operation, a complete external envelope 26 will always be provided on the device 10.

The packages are received at a dispensing site of the medication dispensing device 10 adapted to the type of package 18. In the embodiment shown, package 18 with multiple target locations such as the blister packs shown in FIG. 2, can be positioned in the platen of the interface 20 (or a section thereof) as shown. However, one skilled in the art will understand that the dispensing site may vary. In an alternative embodiment (not shown) a package with a single target location may be received at a dispensing site corresponding to a specific position of the interface 20 having a single target location such as a door, a chute leading to an exposed interface or the like, or to a specific position on a platen configured to receive packages with a single target location.

The structure 12 is the component providing the framework of the device 10 and includes a frame on which all physical components and/or subsystems of the device 10 are mounted. The structure 12 therefore provides a three-dimensional support for the components and/or subsystems of the device 10, which is covered by the external envelope 26 and which defines the footprint of the device 10.

The system controller 24 is the portion of the device 10 which includes instructions stored in a memory of a computing device 25, in data communication with the control electronics 22, to allow the necessary control of the robotic subsystem 16 to perform the dispensing of medications. In the embodiment show, the computing device 25 is a web server and is therefore shown as being outside of the envelope 26 of the device 10, but one skilled in the art will understand that, in an alternative embodiment (not shown) the computing device 25 and the associated system controller 24 could be provided inside the envelope 26 of the device 10. The system controller 24 and operation thereof will be described in more details below.

The interface 20 is provided to allow interaction between users and the device 10. In other words, to retrieve dispensed medications, users can use the interface 20, which is the component of the device 10 which allows the medications to be accessible to the user. As previously mentioned, in an embodiment, the interface 20 can be, doors, tables, conveyors, drawers of similar components for recuperating prepared medication packages, or chutes or holes for recuperating medication units directly. In an embodiment, the interface 20 also allows users to access cassettes 28, for refilling the cassettes 28 with medication units or providing new cassettes to the device 10.

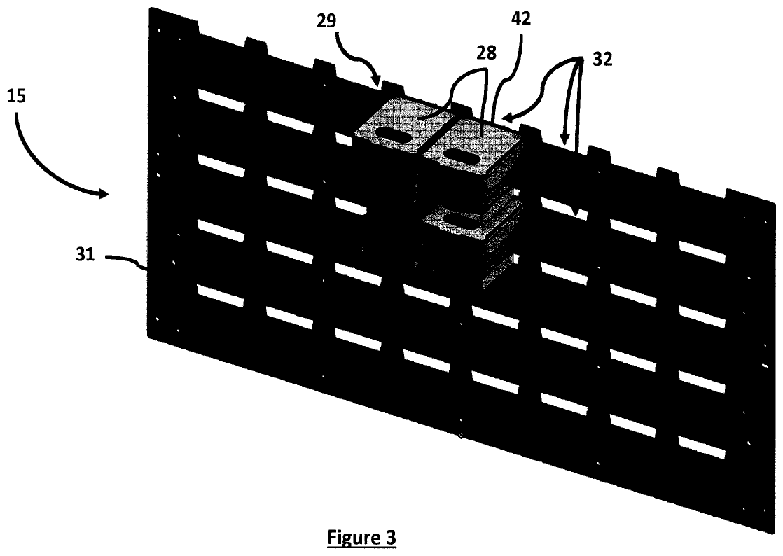

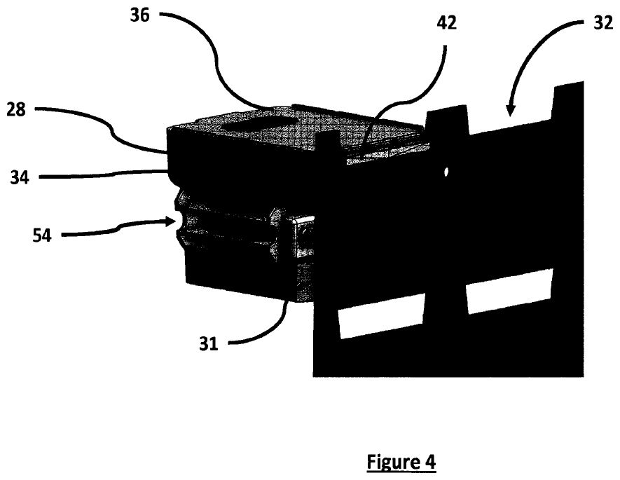

Referring to FIGS. 2, 3 and 4, the cassette storage area 14 is a section of the structure 12 which includes an assembly for receiving a plurality of removable cassettes 28, each holding medication units. In FIG. 2, the cassette storage area 14 is shown with a single cassette 28 being engaged by the robotic head 68 and with a portion of one of the cassette receiving walls 31 removed, to better show the other components of the device 10. FIGS. 3 and 4 show portions of a cassette receiving wall 31 of the cassette storage area 14 with cassettes 28 removably mounted thereon. One skilled in the art will understand that, in operations, a plurality of cassettes 28 will be provided along the cassette storage area 14 of FIG. 2 (i.e. mounted onto the cassette receiving walls 31).

In the embodiment shown, the cassette storage area 14 includes two opposed cassette support rack sections 15 each configured to support at least a subset of the plurality of cassettes 28. The subset of the plurality of cassettes of each cassette support rack sections 15 can be defined as an array of cassettes 29 oriented according to a similar orientation. The two opposed cassette support rack sections 15 would therefore hold two arrays of cassettes having a different orientation (i.e. having opposed orientations as they face each other). As mentioned above, in FIG. 2, a portion of one of the cassette support rack sections 15 is not shown (i.e. had been removed) to better show other components of the device. One skilled in the art would understand that different configurations could be provided in alternative embodiments.

Each cassette support rack section 15 includes a cassette receiving wall 31 having a plurality of slots 32 defined therein. The plurality of slots 32 are each sized and shaped to receive a hooked shaped protrusion of a corresponding cassette, which will be described in more details below, to support the cassette 28 onto the cassette support rack section 15. The hooked shaped protrusion of the corresponding cassette is inserted in the slot and rearwardly engages with a surface of the cassette receiving wall 31 while being supported on a lower edge of the slot 32, to support the cassette 28 on the cassette receiving wall 31. In the embodiment shown, the plurality of slots 32 each have a trapezoid shape, with the narrower base being positioned at the bottom, to allow the hooked shaped protrusion of a corresponding cassette to be moved into place by gravity, when being lowered into the slot 32.

As can be seen in FIG. 3, the specific arrangement of the cassette receiving wall 31 allows the cassette receiving wall 31 to receive and support cassettes 28 of different dimensions.

In the embodiment shown, the cassette support rack sections 15 of the storage area 14 are positioned along opposed longitudinal peripheral sections of the structure 12 (extending along the width of the device), which provides the greater length and surface for the cassette support rack sections 15 and the corresponding arrays of cassettes 29. One skilled in the art will however understand that, in alternative embodiments (not shown), the cassette support rack sections 15 could be arranged in different configurations. The cassette support rack sections 15 could in different orientations and therefore provide arrays of cassettes 29 each providing cassettes 28 having a specific orientation (e.g. arrays of cassettes 29 being perpendicular to one another (e.g. positioned along perpendicular peripheral sections of the structure), in a reversed orientation (e.g. positioned along opposed peripheral section of the structure) or angled with regard to one another).

One skilled in the art will understand that, in alternative embodiments (not shown) the cassette support rack sections 15 of the cassette storage area 14 could provide arrangement for supporting the cassettes 28 such as, for example and without being limitative shelves or guiding rails for receiving the plurality of removable cassettes 28.

In an embodiment (not shown), the cassette storage area 14 could for example include a plurality of guiding rails each projecting inwardly from the structure 12 and defining cassette receiving sections sized and shaped for receiving a cassette 28 and engaging with opposed sides of the cassette 28 positioned therein, to support the cassette 28.

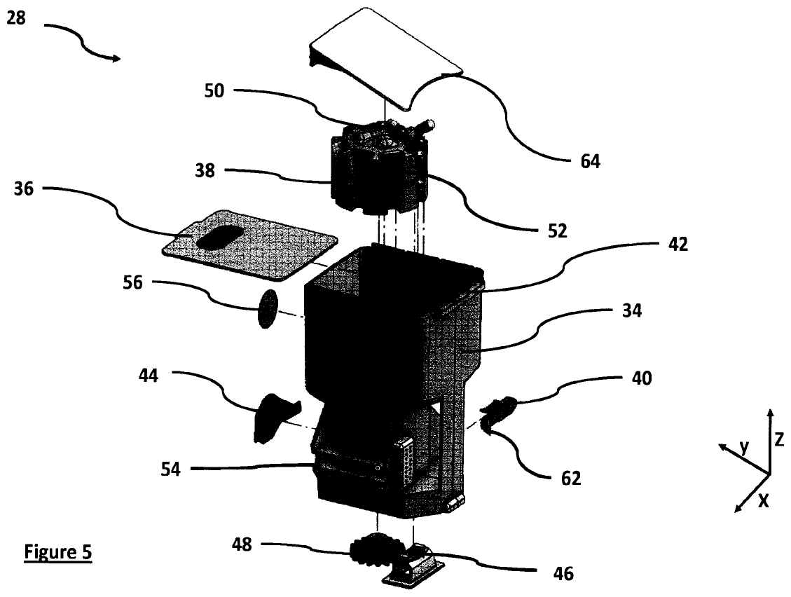

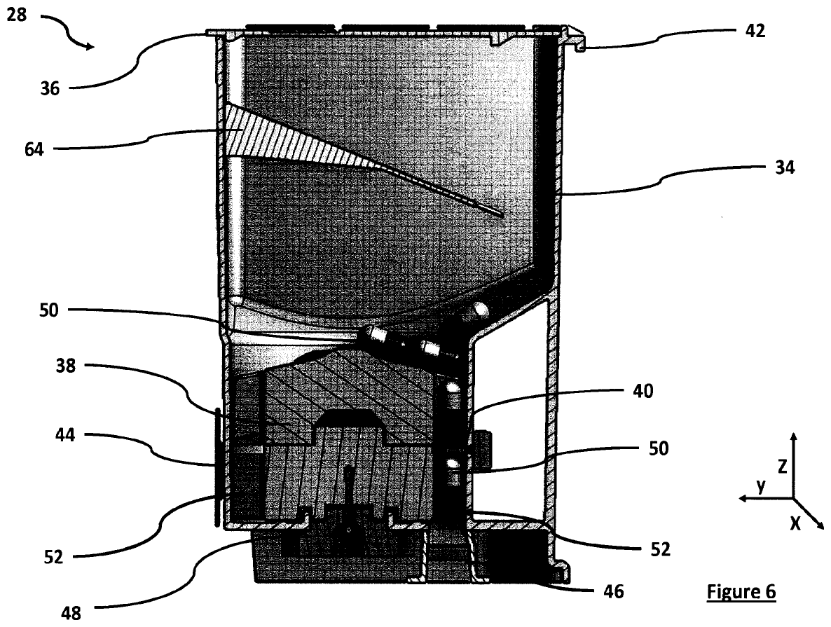

Now referring to FIGS. 5 and 6, an embodiment of the cassettes 28 to be used in the device 10 will be described in more details below.

Typically, each cassette 28 contains medication units of one type of medication drug. In an embodiment one or more cassettes 28 may contain the same medication drug to increase the reserve of the corresponding medication drug of the storage area 14.

In the embodiment shown, each cassette 28 has a housing 34 defining an inner cavity for receiving one or more medication units 50 of a specific medication drug and closable using a lid 36. The lid 36 therefore closes the inlet of the inner cavity defined at the top of the housing 34. The housing 34 may differ in sizes to accept variable quantities of medication units 50.

In the embodiment shown the housing 34 comprises guide channels 54 on each side of the cassette 28, one to engage with the robotic system 16.

In the embodiment shown, the housing 34 also includes a hooked shaped protrusion 42 projecting outwardly from a rear surface of the housing 34. As mentioned above, the hooked shaped protrusion 42 is configured to be engageable through a slot 32 of a cassette receiving wall 31 of the cassette storage area 14, and to be supported onto the cassette receiving wall 31 with the protrusion 42 being supported on the bottom edge of the slot and the hook (or lip) locking against the surface of the cassette receiving wall 31 opposed to the surface onto which the housing of the cassette 34 is abutted.

In an embodiment, the housing 34 could also comprise a cassette identifier 56 containing the information required for the operator to identify the content drug in the cassette 28, such as, but not limited to, the drug name, the drug strength, the drug UPC number and/or the Drug Identification Number. In an embodiment, the cassette identifier is an RFID tag. In alternative embodiment a different identifier, such as, for example and without being limitative a QR code, containing information relative to the position of the cassette 28 in the storage area 14 can also be provided.

The cassette 28 also includes a slotted disk 38 positioned at a lower section of the inner cavity of the housing 34, a chute 46, and a coupling wheel 48 positioned below the housing 34 and coupled to the slotted disk 38 to rotate simultaneously therewith. In other words, the slotted disk 38 is fixed on the internal side of the bottom wall of the housing 34 with the coupling wheel 48 fixed on the external side of the bottom wall of the housing 34. In the embodiment shown, the cassette also includes a deflector 64. The deflector helps preventing accumulation of medication units 50 over the slotted disk 38, as otherwise the friction caused by the weight of the medication units 50 could undesirably hinder the rotation of the slotted disk 38.

The slotted disk 38 has slots 52 defined in the outer periphery thereof, such that medication units 50 can enter the slots 52 on the outer periphery of the slotted disk 38 by gravity. Upon rotation of the slotted disk 38, the medication units 50 can exit the housing 34 through the chute 46. The chute 46 is an extension of the housing 34 which defines an outlet channel at the bottom of the housing 34 and guides the medication unit 50 as it exits the housing 34. As will be described in more details below, the chute 46 is sized, shaped and configured to reduce the non-guided distance from the housing 34 to the package 18 and therefore prevent (or at least minimize) rebound, which can cause damage and/or contamination of the dispensed medication units 50. In an embodiment the chute 46 includes a flared section with an outlet substantially corresponding to a size of a corresponding package or section of package into which the medication units 50 are to be dispensed to prevent (or at least minimize) rebounds. As will once again be described in more details below, the medication units 50 are therefore dispensed when the robotic subsystem 16 couples with the coupling wheel 48 of the cassette 28 and activates the rotation of the slotted disk 38. In more details, as can be seen in FIG. 6, a medication unit 50 is dispensed when a slot 52 of the slotted disk having a medication unit 50 inserted therein aligns with the cassette chute 46 defining the outlet channel of the cassette housing 34.

In order to control the dispensing of the medication units 50, the cassette 28 also includes a retaining member 40. The retaining member 40 is positioned over the outlet channel defined by the chute 46 at the bottom of the housing 34 and controls the quantity of medication unit 50 being dispensed. More precisely, as the dispensed medication unit 50 drops from the cassette housing 34 through the channel of the chute 46, the retaining member 40 prevents another medication unit 50 from exiting the chute 46 after the medication unit 50 creates a temporary empty slot 52. The retaining member 40 is positioned to extend across the Z-axis of the housing 34 and is sized and positioned to fit with the medication unit 50 height when dispensed. In one embodiment, the retaining member 40 is a brush attached with a subassembly of a clamp 62 and fastener.

The cassette locking member 44 is used by the robotic system 16 to lock the cassette 28 on the robotic system 16. In the embodiment shown, the cassette locking member 44 is a magnetic member. One skilled in the art will understand that in alternative embodiments, alternate means could be used to lock the cassette 28 on the robotic system 16 when extracting the cassette 28 from the corresponding cassette receiving wall 31 and unlocking the cassette 28 when returning it on the corresponding cassette receiving wall 31 in the storage area 14. For example and without being limitative, in alternative embodiments, the locking mechanism 44 could be a hook, a gripper, a lock, a rotating mechanical linkage, a holed bracket enabling the insertion of a locking pin, etc.

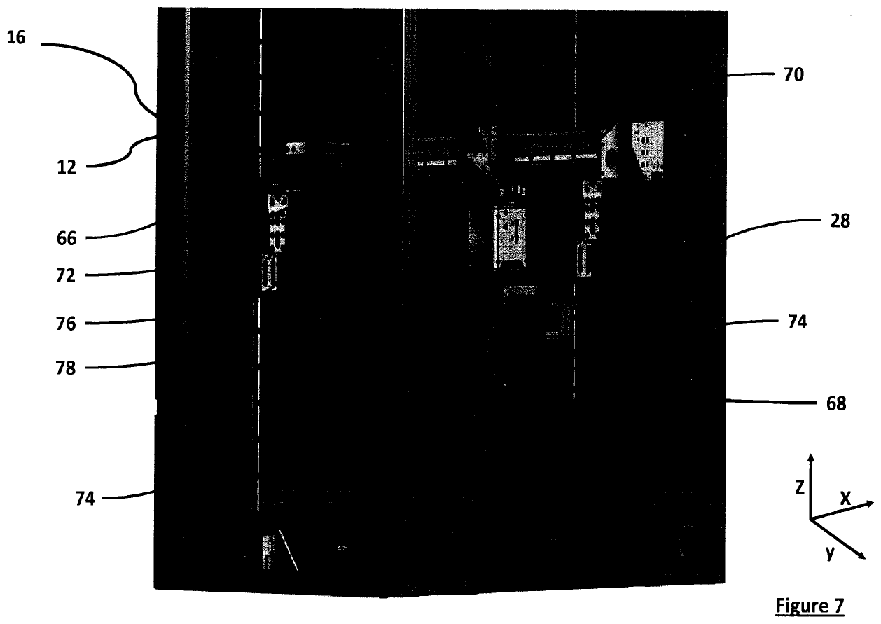

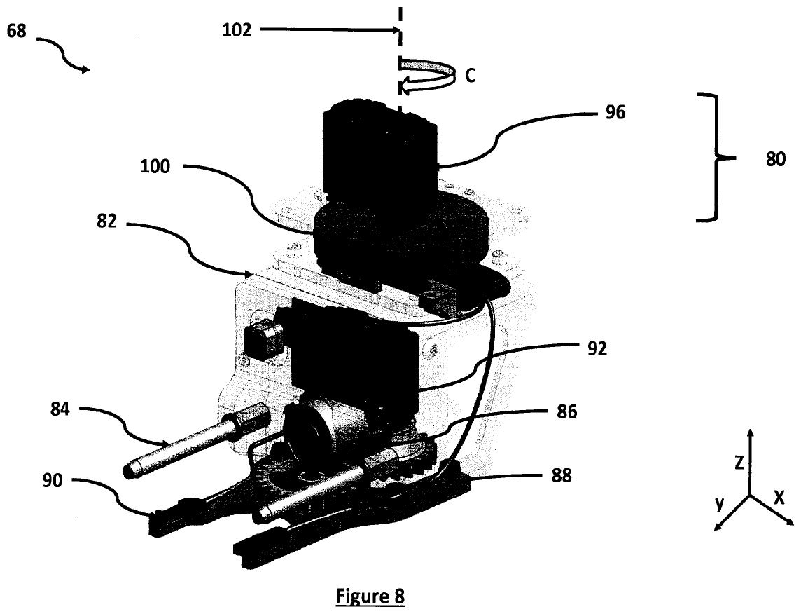

Referring to FIGS. 7 and 8, an embodiment of the robotic subsystem 16 and the corresponding robotic head 68 of the device 10 will now be detailed. The robotic subsystem 16 comprises one or more robotic head 68 and is controlled by the control electronics 22. The control electronics 22 receives a command from the system controller 24 which commands the control electronics 22 to operate the robotic subsystem 16.

As mentioned above, in an embodiment, the system controller 24 is a software or an application. In other words, the system controller 24 includes instructions stored in a memory of a computing device 25 in data communication with the control electronics 22 such that data is exchanged between the control electronics 22 and the system controller 24. The system controller 24 issues system commands, which are communicated to the control electronics 22 controlling the robotic subsystem 16 to perform the system commands and the corresponding dispensing of medication units. In an embodiment, the system controller 24 is an online application, i.e. the system controller 24 is embodied with instructions stored in a memory of a computing device 25 in data communication with the control electronics 22 via a network 27. As mentioned above, alternatively, in an alternative embodiment (not shown), the system controller 24 can includes instructions stored in a memory of a local computing device, i.e. a computing device embedded into the medication dispensing device 10.

In the embodiment shown, the robotic subsystem 16 comprises a motorized system 66 mounted to the structure 12 and a robotic head 68 being moved about the structure by the motorized system 66. In other words, the robotic head 68 is moved to variable spatial positions along the structure 12, through movement of the motorized system 66 to engage and capture a cassette from the cassette storage area 14, move the cassette to at least one target location where medication is dispensed in a specific package 18 and/or store a cassette in a corresponding cassette receiving section of the cassette storage area 14. In an embodiment, the robotic subsystem 16 also allows the cassette 20 to be provided to a user, through the interface 20, for example for refill or replacement.

In the embodiment shown, the motorized system 66 includes a combination of rotative actuators, pulley, timing-belt and roller system for moving the robotic head 68 along guiding rails of the structure 12. One skilled in the art will however understand that, in alternative embodiments, other motorized components such as, for example and without being limitative, linear actuators or the like, could be used for the motorized system 66.

In the embodiment shown, the motorized system 66 includes an actuator 76 associated to each one of an X-axis 70 beam, Y-axis beam 72 and Z-axis beams 74. In other words, each one of the X-axis 70 beam, Y-axis beam 72 and Z-axis beams 74 comprises an actuator 76 and the associated components to move the corresponding portion of the motorized system 66 therealong, such that the motorized system 66 is operative to displace the movable sub-assemblies in the Cartesian system XYZ. In the embodiment shown, the Y-axis beam 72 is mounted on the X-axis beam 70 and is movable along the X-axis beam 70, with the X-axis beam 70 being fixed at each end to one of the Z-axis beams 74 and being movable along the Z-axis beams 74. The Z-axis beams 74 are fixed to the frame of the structure 12. The robotic head 68 is mounted on a carrier 78 which is movable along the Y-axis beam 70.

In an alternative embodiment (not shown), the X-axis beam 70 could be mounted at each end to Y-axis beams 72 and be movable along the Y-axis beams 72, with the Y-axis beams 72 being each fixed at a middle point along the axis to one of the z-axes 74 and being movable along the Z-axis beams 74 fixed to the structure frame 12 and the robotic head 68 being mounted on a carrier 78 which is movable along the X-axis beam 70. In another alternative embodiment (not shown). In other alternative embodiments (not shown), the motorized system 66 could have a single Y-axis beam 72 and/or Z-axis beam 74, or more than two Y-axis beams 72 and/or Z-axis beams 74. In another alternative embodiment, the robotic subsystem 16 could be a robotic arm allowing movement along at least the axes of the Cartesian system XYZ, with the robotic head 68 being the end effector thereof.

Referring more specifically to FIG. 8, in the embodiment shown, the robotic head 68 comprises a rotation mechanism 80, a body 82, a locking mechanism 86, a motorized activation wheel 88, an optical sensor 90 and a cassette identification scanner 92.

The rotation mechanism 80 of the robotic head 68 includes a bearing assembly 100 positioned between the carrier 78 and body 82 of the robotic head 68, to allow rotation of the body 82 of the robotic head 68 with regard to the carrier 78 and positioning of the robotic head 68 along a C-axis. The rotation mechanism 80 further includes an actuator 96 activating the bearing assembly 100 to rotate the body 82 of the robotic head 68 around the vertical axis 102, so that the body 82 can face a specific direction. In an embodiment, the assembly is a direct drive assembly between the actuator 96 and the bearing assembly 100. However, one skilled in the art would understand that, in alternative embodiments, alternative assemblies could be provided to allow rotation of the body 82 of the robotic head 68 around the vertical axis 102 (along the C-axis). One skilled in the art will understand that, in an alternative embodiment (not shown), the rotation system 80 could be embodied by a rotary actuator or any mechanical system such as, but not limited to, a gear system, a direct drive system, a cam, a lever system or the like.

In the embodiment shown, the body 82 can be rotated to face different specific directions when the storage area 14 is configured to present one or more cassettes 28 in a different orientation, such as, but not limited to, non-parallel walls, a carousel or the like.

One skilled in the art will also understand that, in alternative embodiments (not shown), the robotic subsystem 16, including the motorized system 66 and the robotic head 68 could include different, additional or fewer degree of freedoms than those of the embodiments shown.

The body 82 includes head guiding rails 84, matching the corresponding cassette channels 54 of the cassette 28, to receive and support a cassette 28. In one embodiment, the head locking mechanism 86 is an electromagnet which is activable to be secured against the cassette locking member 44 being a magnetic member for removably coupling the cassette 28 to the robotic head 68. It will be understood that the head locking mechanism 86 matches the cassette locking member 44, which can consist of, without being limited to, a hooking system, a magnet system, a gripper system, a lock system or a rotating mechanical linkage system, etc. Hence, for example and without being limitative, in an alternative embodiment (not shown), the head locking mechanism 86 could be embodied by a pin at the end of the electromagnet that can be inserted in a corresponding cassette locking member 44.

The head guiding rails 84 and the head locking mechanism 86 allow the robotic head 68 to engage with a cassette and to remove the cassette from the cassette storage area 14 for subsequent displacement to a corresponding spatial target location where medication units are to be dispensed.

The robotic head 68 is configured such that when the cassette 28 is locked onto the robotic head 68, the motorized activation wheel 88 is engaged with the coupling wheel 48 of the cassette 28. Therefore, to perform the dispensing of the medication units 50 into a package 18, a corresponding cassette 28 is locked on the robotic head 68, the robotic head 68 and the cassette 28 locked onto it are moved along the Cartesian plan (i.e. along the X, Y and Z axis) to a target position for dispensing of the medication units into the package 18 by the motorized system 66, and the activation wheel 88 is subsequently rotated to rotate the coupling wheel 48 (which in turn rotates the slotted disk 38 inside the cassette 28 and guides medication units towards the chute 46). In an embodiment, the robotic head 68 and the cassette 28 locked onto it can also be moved along the C-axis to the target position.

In an embodiment, the robotic head 68 includes an actuator 87 operative to rotate the activation wheel 88, for example via a gear mechanism or any other transmission system, to perform rotation of the activation wheel 88.

The presence of the activation wheel 88 on the movable robotic head 68 is highly advantageous. For example and without being limitative, it allows the dispensing of one or more medication units 50 from a cassette 28 at a first target location where medication is dispensed in a specific package 18 or a specific section of a package 18, and subsequent displacement of the robotic head 68 towards one or more additional target locations where medication is dispensed in another specific package 18 or a specific section of a package 18 and dispensing of the medication units 50 from the cassette 28 at each one of the additional target locations (by rotating the activation wheel 88), without requiring specific components for performing the dispending of the medication units from the cassette at each target locations. This allows the device 10 to be highly versatile, have a high throughput and the footprint of the device to be minimized, while allowing the multiple target locations to be associated with different container types for the packages 18. The coupling and activation of a cassette 28 and the positioning of the robotic head 68 along all axes of the Cartesian plan, including the Z-axis, allows controls over the distance of a dispensed medication unit drop from the cassette to the package, which helps to prevent (or at least minimize) rebound of the medication unit 50, which is critical to avoid contamination and/or damage.

In one embodiment, an optical sensor 90 is positioned under the cassette chute 46, when a cassette is locked onto the robotic head 68, to perform the counting of the delivered quantity of medication units 50, as they are released from the cassette 28 through the cassette chute 46. One skilled in the art will understand that, in alternative embodiments, a different type of sensor allowing sensing of the dispensed and the corresponding delivered quantity of medication units 50 could be used.

The cassette identification scanner 92 fixed on the body 82 of the robotic head 68 is used to identify an ID of the cassette 28 in proximity of the robotic head 68. For example and without being limitative, in the embodiment shown, the cassette identification scanner 92 is a RFID scanner scanning a RFID tag of the cassette 28. In another embodiment (not shown), the cassette identification scanner 92 could be a barcode scanner 92 used to retrieve a cassette number on a cassette identification label.

In an embodiment, the robotic subsystem 16 also allows a cassette 28 to be guided to the interface 20 of the device, where the cassette 28 can be refilled with medication units 50 (or be exchanged for a different filled cassette 28). The robotic subsystem 16 is configured to subsequently guide the cassette to the corresponding position along the structure 12, and the robotic head 68 is configured to position the cassette at the designated position along the corresponding cassette receiving wall 31 of the cassette storage area 14 and subsequently disengage therefrom.

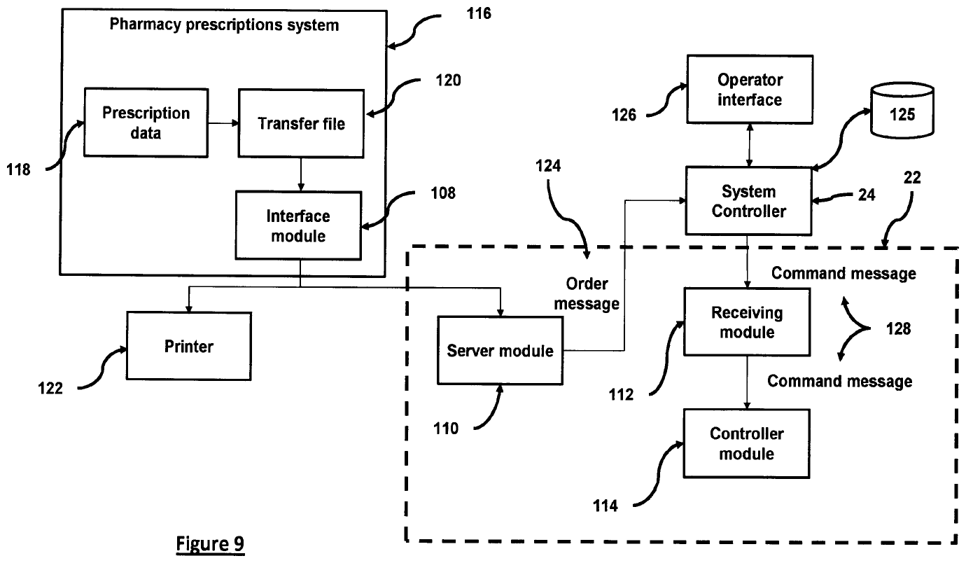

Now referring to FIG. 9, a communication system of the medication dispensing device 10, in accordance with an embodiment is shown. In the embodiment shown, the control electronics 22 of the apparatus 10 operate in cooperation with an interface module 108 which may be installed on a pharmacy prescription management system 116. The control electronics 22 can include, among other components, a server module 110, a receiving module 112 and a controller module 114. The medication distribution command defining the medication units to be dispensed and the quantity thereof can originate from the pharmacy prescriptions system 116. One embodiment of the transfer of the prescription data 118 to the device 10 can be through an integrated module in the pharmacy prescription system 116. In another embodiment, the prescription data 118 can be transferred to the device 10 when one operator performs an action in the pharmacy prescription system 116 which creates a data file such as a data transfer or printing a file. The transfer file 120 may be, among others, an invoice, a drug label, a container label, a vial label or a blister-card sheet.

In one embodiment, when the transfer file 120 is created by a print request from the operator, it is sent to the interface module 108. In an embodiment, the interface module 108 is a module configured to dispatch the printing file 120 to a prescribed printer 122 in the pharmacy and to the server module 110. The interface module 108 receives the transfer file 120, in a specific language, and may or may not modify or alter the printing file 120 content. The printer 122 of the pharmacy may or may not print the physical file. The server module 110 is a module configured to extract content from the transfer file 120 and analyze the prescription data 118. In one embodiment, the interface module 108 and the server module 110 may be integrated into a single module and have additional functions. When the server module 110 has analyzed the prescription data 118, an order message 124 with the information, partial or complete, from the prescription data 118 can be sent to the system controller 24. As previously mentioned, in the embodiment shown, the system controller 24 is stored in a computing device in data communication with the device 10 through a network, but in alternative embodiment, it could be embodied on a local computing device of the device 10. The order message 124 may be sent using any suitable transport or message protocols.

The system controller 24 is in data communication with data sources 125 storing data relative to, without being limitative, a list of possible medication drugs, a list of available medication drugs currently found in cassettes 28 stored in the cassette storage area 14, cassette identifiers for each cassette storing a corresponding medication drug, etc.

The system controller 24 is configured to interpret the order message 124 and generate a command message 128 sent to the receiving module 112 defining the medication units to be dispensed and how they are to be dispensed. During this operation, the system controller 24 can be configured to modify the received order message 124. For example and without being limitative, n one embodiment, the original prescription data 118, such as the Drug Identification Number (DIN), can be modified so that the DIN is changed to a specific cassette identifier found in the above-described data sources 125 accessible to the system controller 24.

In an embodiment, the system controller 24 can have a front-end interface accessible with an operator interface 126. In one embodiment, the operator interface 126 is a graphical user interface displayed on a display unit of a computing device such as, for example and without being limitative, a desktop computer, a laptop, a cellphone, etc. In an alternative embodiment, the operator interface 126 could also be a graphical user interface displayed on a display unit of the device 10 and used with corresponding input means of the device 10.

In an embodiment, an operator can therefore use the operator interface 126 to input data sent to the system controller 24 (e.g. to modify an order message or generate an order message 124) which subsequently creates a command message 128 that is sent to the receiving module 112 of the control electronics 22. The operator interface 126 can also be used by the system controller 24 to communicate information to an operator of the device, as will be described in more details below.

One skilled in the art will understand that, in an embodiment, specific medication drug may not be communicated in the command message 128 if the system controller 24 identifies that the specific medication drug is not stored in any cassette 28 of the cassette storage area 14 (and therefore not listed in the list of available medication drugs stored in the cassette storage area 14 of the data sources 125 accessible to the system controller 24). In one embodiment, the system controller 24 can display a message relative to the missing medication drug being part of the order message 124, but removed from the command message 128, for example through the operator interface 126 or by printing the information on the corresponding package label. The operator can then know what medication unit(s) 50 is/are required to be added to the package 18 manually or using another method of distribution. In an embodiment, the system controller 24 can also verify if the medication units 50 of each cassette 28 listed in the order message 124 are in sufficient quantity to complete the full distribution command prior to sending of the command message 128. In an embodiment, the system controller 24 can further verify different information related to the integrity of the medication units 50 available such as, but not limited to, the expiration date or use-by date. The system controller 24 can display alerts or possible errors of any kind via the operator interface 126, to inform the operator accordingly.

The receiving module 112 transfers the command message 128 to the controller module 114. The controller module 114 is configured to process the command message 128 and to control the robotic subsystem 16 in order to perform the required distribution of the requisite quantity of medication units, each at an appropriate target location to be dispensed in the proper medication package 18 or section of the medication package 18. In another embodiment, the receiving module 112 and controller module 114 can be merged into a single module.

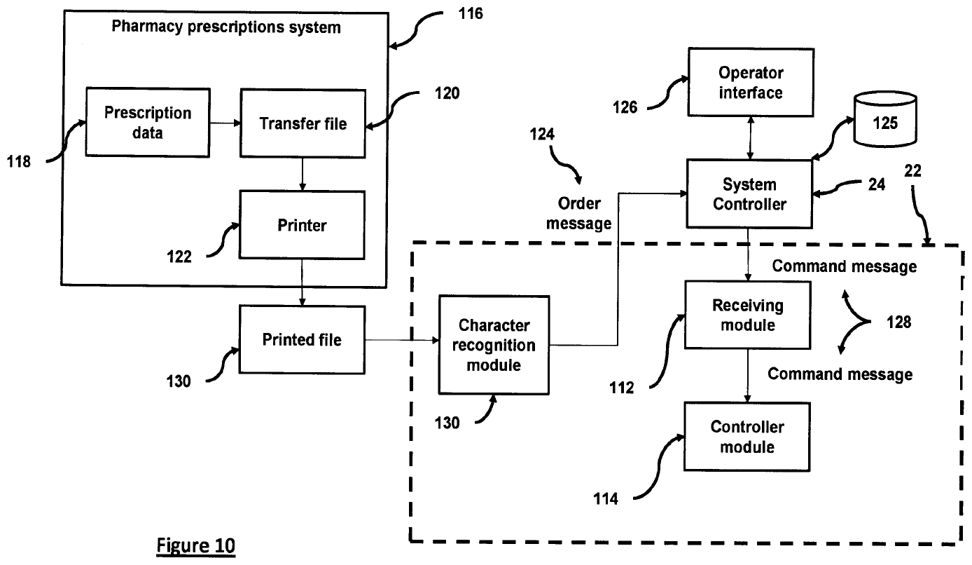

FIG. 10 shows a communication system of the medication dispensing device 10, in accordance with an alternative embodiment. One skilled in the art will understand that the embodiments shown in FIGS. 9 and 10 are not mutually exclusive and could both be embodied simultaneously. In the embodiment of FIG. 10, the transfer file 120 is printed by a printer 122 of the pharmacy creating a printed file 130. In the embodiment shown, the communication system includes a character recognition module 132 configured to analyze the content of the printed file 130. The character recognition module 132 analyzes the prescription data 118 contained in the printed file 130, using one or more character recognition system, and transfers an order message 124 to the system controller 24. In an embodiment, the character recognition module 132 can also be used only for patient identification, for example when the prescription data is already transferred to the system controller 24. Once again, the order message 124 may be sent using any suitable transport or message protocols. The system controller 24, operator interface, 126 receiving module 112 and controller module 114 operate similarly to the embodiment described in relation to FIG. 9 and the functioning and characteristics thereof will not be repeated herein for ease of description.

The medication dispensing device having been described in detail below, the corresponding method for dispensing the medication unit will now be described.

Summarily described, the method includes steps of selecting a cassette from the cassette storage area, coupling the selected cassette to one or more robotic heads, moving a cassette with a robotic head to a dispensing site and activating the cassette with a robotic head over a target location to dispense a selected quantity of medication units. As will be understood, the package can be of different types and/or can have multiple target positions to receive one or more medication units which require a robotic head to perform additional incremental displacements and access all possible target positions for a corresponding package. In accordance with the present method, one or more target positions, such as a single target location or a multiple locations array for a corresponding package may be filled with one or more medication units dispensed from a cassette coupled and activated by a robotic head. The method includes the coupling and activation of a cassette and the positioning of the robotic head in the regards of all axes of the Cartesian plan (XYZ). In an embodiment, the method can also include positioning of the robotic head along the C-axis. The positioning includes positioning along the Z-axis to limit the distance of a dispensed medication unit drop from the cassette to the target position.

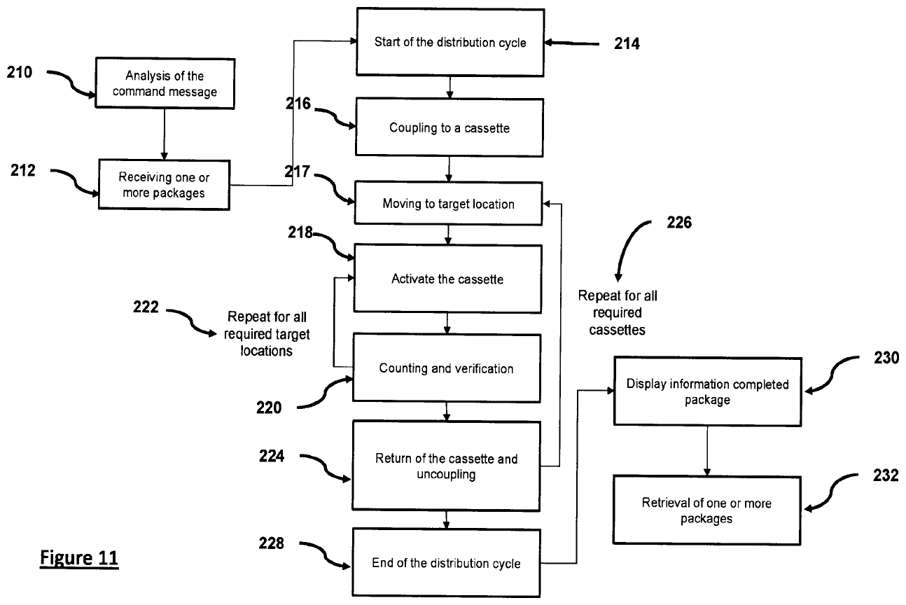

FIG. 11 presents a flowchart with detailed steps of the method 200, in accordance with an embodiment.

In FIG. 11, at step 210, the method includes receiving and analyzing a command message 128. The command message 128 includes minimally instructions relative to one or more cassettes including medication units to be dispensed, a package type or location associated with at least one spatial target locations reachable by the robotic subsystem 16 and one or more quantities of medication units 50 to be dispensed at each one of the at least one spatial target location corresponding to a package or a section of a package onto which medication units are to be dispensed.

At step 212, the method includes the step of receiving one or more packages at a dispensing site of the medication dispensing device adapted to the type of package. Indeed, depending on the type of package, the dispensing site may vary. A package with a single target location may be received at a dispensing site corresponding to a specific position of the interface 20 having a single target location such as a door, a chute leading to an exposed interface or the like, or to a specific position on a platen configured to receive packages with a single target location. A package with multiple target locations may correspond to a platen of the interface 20 (or a section thereof). At this step one or more packages 18 can be received, each package being received at a corresponding dispensing site.

Step 214 defines the start of the distribution cycle.

At step 216, the method includes the step of coupling of the robotic head 68 with cassette 28. In more details, during this step, the robotic subsystem 16 engages with a cassette 28. More particularly, the robotic head 68 of the robotic subsystem 16 engages a cassette 28 stored in the cassette storage area 14 and retrieves the cassette 28 from the cassette storage area 14. In an embodiment, the step of retrieving the cassette 28 from the cassette storage area 14 includes moving the cassette 28 with the robotic head 68 to disengage the hooked shaped protrusion 42 projecting outwardly from the rear surface of a housing 34 of the cassette 28 from a slot 32 of the cassette receiving wall 31 of the cassette storage area 14.

During this step, the robotic subsystem 16 can perform a cassette identification verification using the cassette identification scanner 92, to validate that the proper cassette 28 is being retrieved.

At step 217, the method includes the step of moving of the cassette to a target location. This step includes moving the cassette 28 to a first spatial target location for dispensing at least one medication unit 50 to a specific package or section of a specific package. In an embodiment, the first special target location is defined in the command message 128. In an embodiment, this step includes positioning the cassette 28 along the Z-axis by the robotic subsystem 16, to minimize rebounds and/or contamination of the medication unit 50 being dispensed therefrom.

At step 218, the method includes the step of activating the cassette 28 for performing distribution of at least one medication unit. The distribution starts after the robotic head 68 has been moved by the robotic subsystem 16 to position the cassette 28 in the spatial location corresponding to at the first spatial target location for a specific package or section of a specific package. The robotic head 68 activates the cassette 28 over the first spatial target location for the corresponding package 18. More particularly, the robotic head 68 rotates the activation wheel 88 thereof, which in turn rotates the coupling wheel 48 and the slotted disk 38 of the cassette 28 until a requested quantity of medication units 50 for the specific package or section of the specific package to has been dispensed. The quantity of medication units being dispensed at the first target location is instructed in the command message 128.

At step 220, the method includes the counting and verification step. In this step a system verifies the dispensed medication unit quantity. Another system may verify the type of medication dispensed or take a picture for future approbation, traceability or verification. In an embodiment, this step may include a “container-content” verification.

At step 222, repetition of steps 217, 218 and 220 is performed with the selected cassette 28 until all target locations requiring a certain quantity of the medication units in the cassette 28 are dispensed. In one embodiment, the package 18 may have one target location. In another embodiment, the package 18 may have multiple target locations, such as a matrix of target locations. In yet another embodiment, a plurality of single location target packages may be used, with multiple target locations requiring a certain quantity of the medication units in the cassette 28 being required to provide the medication units to the plurality of single location target packages.

Therefore, during this step 222, the robotic head 68 is moved such that the selected cassette 28 is sequentially moved to all required target locations for providing the medication units into the associated package(s) 18 and the robotic head 68 activates the cassette 28 accordingly to perform the dispensing of the medication unit at the at least one target location. In an embodiment, this step also includes positioning the cassette along the Z-axis for the at least one target location, to minimize rebounds of the medication unit being dispensed therefrom.

Step 224 concerns the return of the cassette 28 into the cassette storage area 14. At this step, when the medication units 50 distribution is completed for the selected cassette 28, the robotic head 68 returns the cassette 28 to its associated position in the cassettes storage location 14 and uncouples the cassette, thereby freeing the cassette 28 therefrom. In an embodiment, this step includes moving the cassette 28 with the robotic head 68 to engage the hooked shaped protrusion 42 projecting outwardly from the rear surface of the housing 34 of the cassette 28 into the slot 32 of a cassette receiving wall 31 of the cassette storage area 14.

Step 226 concerns selection of the next cassette if needed. Upon normal use, the process of steps 216, 217, 218, 220, 222 and 224, is repeated for the entire list of cassettes (and corresponding medication drugs) included in the command message 128. The robotic head 68 therefore moves and couples to each required cassette 28 listed in the command message 128 to perform dispensing of the corresponding medication in accordance with steps 216, 218, 220, 222 and 224 at all requested spatial target locations.

Step 228 defines the end of the distribution cycle. The process ends when all cassettes from the command message 128 have been used for distribution of the associated medication units, or if the process is stopped by an operator. In one embodiment, once the package is completed, the package 18 can be labelled by a packaging and labelling system part of the device 10.

Step 230 defines the display of information relative to a completed package. For this step, indication that the distribution cycle is finished can be performed by any means, such as text on a screen, light indicator, etc. During this step, information relative to the medication units of the package or the associated patient can also be displayed. For example, the operator interface 126 can be used to display the information relative to the medication units of the package or the associated patient.

Step 232 concerns the retrieval of one or more filled package 18, for example by the operator or a robotic mean. The operator can interact with the interface 20 and expose one or more package 18. The operator can then retrieve a platen supporting one or more packages 18 having been filled. In another embodiment, one or more vial 138 could be supported by a mechanical component during dispensing inside the envelope 26. In another embodiment, one or more vial 138 can be supported by the operator during dispensing with the package 18 inside or outside the envelope 26 via an exposed chute. When one or multiple filled package 18 are available, the interface system 20 or any visual indicators can lead the operator to the selected package 18. In another embodiment, the medication units 50 are dispensed in a packaging system which can include a packet packaging system.

One skilled in the art will understand that the method can include additional steps associated with features of the device 10 described above. In fact, one skilled in the art would understand that steps corresponding to functioning of the features of the device are considered to be included in the method.

For example and without being limitative, in an embodiment, the method can include the step of activating the head locking mechanism 86 of the robotic head 68 to secure the head locking mechanism 86 with a cassette locking member 44 of the cassette 28, such that the motorized activation wheel 88 is engaged with the coupling wheel 48 of the cassette 28 when the cassette locking mechanism 86 is secured with the cassette locking member 44. In an embodiment, this can include activating an electromagnet of the robotic head to secure the electromagnet against a magnetic member of the cassette.

In an embodiment, the method can also include the step of rotating a body 102 of the robotic head 68 along a C-axis, so the body can face a specific direction for engaging the cassettes 28 of a corresponding one of at least two arrays of cassettes of the cassette storage area 14 each having a different orientation.

The device 10 and method described hereinabove provides significant advantages. For example, the device has a significant productivity and cassette capacity with minimal footprint and no cross-contamination during dispensing of medication units. The cassette storage architecture and position can provide increased design configuration since the robotics perform the moving and dispensing cycle operations. Moreover, a cassette does not require an integrated energized system which reduces its size and increases the possibility of the apparatus cassettes capacity. Furthermore, the packages do not have to be moved, which reduce the possible footprint of the apparatus and some medication distribution operations are performed by a versatile robotic head reducing the quantity of present systems, therefore reducing the possible footprint or increasing the cassette capacity. The method and system also prevent medication units dispensing errors, such as medication unit rebounds, by reducing the medication unit drop height and, in parallel, can reduce the cassette chute and size which increases the cassette capacity of the device. Furthermore, a plurality of packages or a plurality of package types can be used with one or more spatial dispensing target locations which are accessible by the robotic head directly including the necessary means for activating the dispensing of medication units from a cassette engaged thereto. Indeed, the device 10 and method described hereinabove allow several different types of packages (e.g. vials and blisters to be used in the same device). This allows large possibilities of dispensing functions and package types providing more automated activities normally performed by human resources.

Several alternative embodiments and examples have been described and illustrated herein. The embodiments of the invention described above are intended to be exemplary only. A person of ordinary skill in the art would appreciate the features of the individual embodiments, and the possible combinations and variations of the components. A person of ordinary skill in the art would further appreciate that any of the embodiments could be provided in any combination with the other embodiments disclosed herein. It is understood that the invention could be embodied in other specific forms without departing from the central characteristics thereof. The present examples and embodiments, therefore, are to be considered in all respects as illustrative and not restrictive, and the invention is not to be limited to the details given herein. Accordingly, while the specific embodiments have been illustrated and described, numerous modifications come to mind. The scope of the invention is therefore intended to be limited solely by the scope of the appended claims.

1. A medication units dispensing device comprising:
a cassette storage area for storing at a plurality of cassettes;
the plurality of cassettes stored in the cassette storage area, each one of the plurality of cassettes storing medication units of a medication drug and including a housing, a chute defining an outlet channel of the housing, a slotted disk positioned at a lower section of the housing with slots being defined in a periphery of the slotted disk to receive medication units being guided therein by gravity and a coupling wheel positioned under the housing and engaged with the slotted disk to rotate simultaneously with the slotted disk, wherein the slotted disk is rotatable to move the medication units received in the slots towards the outlet channel of the chute and individually dispense the corresponding medication units through the chute;
a robotic subsystem including a motorized system and a robotic head movable about at least an X-axis, a Y-axis and a Z-axis by the motorized system, the robotic head being removably engageable with each one of the plurality of cassettes to remove a corresponding one of the cassettes from the cassette storage area and move the corresponding one of the cassettes to at least one target location where at least one of the medication units can be dispensed in a specific package or a specific section of the package, the robotic head including a motorized activation wheel engaged with the coupling wheel of the corresponding one of the cassettes when the robotic head is engaged to the corresponding one of the cassettes and an actuator for rotating the activation wheel to dispense at least one of the medication units at the at least one target location;
control electronics controlling the operation of the robotic subsystem; and
a system controller issuing system commands communicated to the control electronics.

2. The medication dispensing device of claim 1, wherein the robotic subsystem is configured to move the corresponding one of the cassettes to a plurality of target locations where at least one of the medication units can be dispensed in a specific package or a specific section of the package.

3. The medication dispensing device of claim 1, wherein each one of the plurality of cassettes includes a cassette locking member and the robotic head includes a head locking mechanism activable to be secured with the cassette locking member of corresponding one of the cassettes, the motorized activation wheel being engaged with the coupling wheel of the corresponding one of the cassettes when the cassette locking mechanism is secured with the cassette locking member thereof.

4. The medication dispensing device of claim 3, wherein the cassette locking member includes a magnetic member and the head locking mechanism includes an electromagnet activable to be secured against the magnetic member of the corresponding one of the cassettes for removably coupling the robotic head to the corresponding one of the cassettes.

5. The medication dispensing device of claim 1, wherein the cassette storage area includes at least one cassette support rack section configured to support at least a subset of the plurality of cassettes, with the subset of the plurality of cassettes defining an array of cassettes oriented according to a similar orientation.

6. The medication dispensing device of claim 5, wherein the cassette storage area includes at least two cassette support rack sections each configured to support at least a subset of the plurality of cassettes defining an array of cassettes, thereby defining at least two arrays of cassettes, with the cassettes of each one of the least two arrays of cassettes having a different orientation, and wherein the robotic head includes a rotation mechanism rotating a body of the robotic head so the body can face a specific direction for engaging the corresponding of the cassettes being part of any one of the least two arrays of cassettes.

7. The medication dispensing device of claim 5, wherein each one of the plurality of cassettes includes a hooked shaped protrusion projecting outwardly from a rear surface of the housing and wherein each cassette support rack section of the cassette storage area includes a cassette receiving wall having a plurality of slots defined therein, the plurality of slots each being sized and shaped to receive the hooked shaped protrusion of one of the plurality of cassettes and to support the one of the plurality of cassettes onto the cassette support rack section.

8. The medication dispensing device of claim 1, wherein the robotic subsystem is configured to position the corresponding one of the cassettes at a position along the Z-Axis minimizing rebounds of the at least one of the medication units being dispensed from the corresponding one of the cassettes, for each target location.

9. The medication dispensing device of claim 1, wherein the robotic head includes an optical sensor positioned below the chute of the corresponding one of the cassettes engaged with the robotic head, the optical sensor allowing counting of a delivered quantity of the at least one of the medication units dispensed from the corresponding one of the cassettes, through the chute.

10. The medication dispensing device of claim 1, wherein the chute of each one of the plurality of cassettes is sized, shaped and configured to minimize rebounds of the at least one of the medication units being dispensed therefrom.

11. An automated method for medication units dispensing, the method including the steps of:
a) engaging a cassette stored in a cassette storage area with a robotic head of a robotic subsystem and retrieving the cassette from the cassette storage area;
b) moving the cassette to a first spatial target location for dispensing at least one medication unit to a specific package or section of a specific package;
c) activating the cassette at the first target location by rotating an activation wheel of the robotic head, which in turn rotates a coupling wheel and a slotted disk of the cassette, to dispense a quantity of medication units corresponding to a requested quantity for the specific package or section of the specific package;
d) repeating steps b) and c) with the selected cassette for additional target locations, until all target locations corresponding to at least one specific package or section of at least one specific package requiring a certain quantity of the medication units in the cassette are dispensed;
e) returning the cassette to the cassette storage area using the robotic head and disengaging the cassette from the robotic head; and
f) repeating steps a) to e) for additional cassettes storing medication units to be dispensed in the specific at least one specific package or section of at least one specific package.

12. The method of claim 11 including the further step of receiving and analyzing a command message including instructions relative to one or more cassettes including medication units to be dispensed, a package type associated with at least one spatial target position and one or more quantities of medication units to be dispensed at each spatial target locations corresponding to the specific package or section of the specific package.

13. The method of claim 11, further comprising the step of receiving one or more packages at dispensing site, for receiving medication units.

14. The method of claim 11, further comprising the step of, verifying the dispensed quantity of medication units at each target locations using an optical of the robotic head.

15. The method of claim 11, further comprising the step of activating a head locking mechanism of the robotic head to secure the head locking mechanism with a cassette locking member of the cassette, the motorized activation wheel being engaged with the coupling wheel of the cassette when the cassette locking mechanism is secured with the cassette locking member.

16. The method of claim 15, wherein the step of activating a head locking mechanism of the robotic head to secure the head locking mechanism with a cassette locking member of the cassette, includes activating an electromagnet of the robotic head to secure the electromagnet against a magnetic member of the cassette.

17. The method of claim 11, further comprising rotating a body of the robotic head so the body can face a specific direction for engaging the cassettes of a corresponding one of at least two arrays of cassettes of the cassette storage area, each array including a subset of cassettes oriented in a specific orientation, with the orientation being different for each array.

18. The method of claim 11, further comprising positioning the cassette along the Z-axis, in at least one of the first target location and the additional target location, to minimize rebounds of the medication unit being dispensed therefrom.

19. The method of claim 11, wherein the step of retrieving the cassette from the cassette storage area includes moving the cassette with the robotic head to disengage a hooked shaped protrusion projecting outwardly from a rear surface of a housing of the cassette from a slot of a cassette receiving wall of the cassette storage area.

20. The method of claim 19, wherein the step of returning the cassette to the cassette storage areal includes moving the cassette with the robotic head to engage the hooked shaped protrusion projecting outwardly from the rear surface of the housing of the cassette into the slot of a cassette receiving wall of the cassette storage area.
